(19) United States
(12) Patent Application Publication
(10) Pub. No.: US 2006/0247442 A1
(43) Pub. Date: Nov. 2, 2006

(54) Process for preparing isatins with control of side-product formation

(75) Inventors: Bogdan Kazimierz Wilk, New City, NY (US); Panolil Raveendranath, Monroe, NY (US); Zhixian Ding, Fort Lee, NJ (US); Peter Wehrenberg, Plattsburgh, NY (US)

Correspondence Address: HOWSON AND HOWSON;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/413,160

(22) Filed: Apr. 27, 2006

Related U.S. Application Data
(60) Provisional application No. 60/676,149, filed on Apr. 29, 2005.

(51) Int. Cl. C07D 209/36; C07D 405/02; C07D 403/02

(52) U.S. Cl. 548/465; 548/485

(57) ABSTRACT

Methods and kits for preventing or minimizing the formation of isatin oximes during formation of an isatin from an isonitrosoacetanilide are provided. Also provided are methods and kits for preventing or minimizing the formation of isatin oxime impurities after formation of an isatin from an isonitrosoacetanilide by using a decoy agent in the quenching and/or extraction steps. The isatins can be prepared using a decoy agent and desirably a strong acid. Further provided are methods for preparing isatin oximes.

### CROSS-REFERENCE TO RELATED APPLICATIONS

This application claims the benefit of the priority of U.S. Provisional Patent Application No. 60/676,149, filed Apr. 29, 2005.

### BACKGROUND OF THE INVENTION

Methods and kits for preventing or minimizing the formation of isatin oxime impurities after formation of an isatin from an isonitrosoacetanilide by using a decoy agent are provided.

Isatins (1H-indole-2,3-diones) are useful intermediates in the synthesis of oxindoles, 4-carboxyquinolines, and biologically-active substances. The most frequently used route to isatins is that of Sandmeyer (T. Sandmeyer Helv. Chim. Acta 1919, 2, 234) in which an aniline is converted to an anilide of glyoxylic acid oxime which is subsequently treated with a strong acid to close the ring to an isatin. Usually, if not always, the isatin product is accompanied by undesired isatin oxime side-product (M. Kollmar et al., Organic Syntheses, 2002, 79, 196; C. S. Marvel, G. S. Hiers, 1941, Coll. Vol. I, 327; A. H. Gouliaev et al., International Patent Publication No. WO 2004/018466 A2; and F. Piozzi, Chemical Abstracts 51, 1872e (1957); and Atti. accad. nazl Lincei Rend. Classe sci. fis., mat e nat 19, 44-9 (1955)).

The amount of side product in the reaction mixture varies depending on the structure, number of extractions, temperature, and time. The side product usually accounts for 10-50% of the product, especially when the product cannot be precipitated but rather is extracted with an organic solvent. The formation of oxime side-product therefore decreases both yield and purity of the isatin product. A large amount of work is necessary to purify the isatin product from the accompanying oxime side-product. See, for example, Gouliaev, cited above, in which the crude isatin product contained 9-13% of the oxime side-product.

What is needed in the art are methods of preparing and isolating isatins with low levels of side-products.

### SUMMARY OF THE INVENTION

In one aspect, methods for preventing or minimizing the formation of isatin oximes are provided.

In another aspect, methods for preventing or minimizing the formation of isatin oximes during formation of an isatin from an isonitrosoacetanilide are provided.

In a further aspect, methods for preventing or minimizing the formation of isatin oximes after formation of an isatin from an isonitrosoacetanilide are provided.

In another aspect, methods of preparing 7-fluoroisatin are provided.

In still another aspect, methods for preparing an isatin oxime are provided.

Other aspects and advantages of the invention will be readily apparent from the following detailed description of the invention.

### BRIEF DESCRIPTION OF THE FIGURES

FIG. 1 provides a graph of the production of isatin oxime over time using the procedure set forth in Example 4.

FIG. 2 provides a graph of the efficiency of extracting the product of Example 6, as a function of area count, using ethyl acetate (♦) or a mixture of ethyl acetate and acetone (▪) using 1, 2, and 3 extractions.

FIG. 3 provides a graph of the efficiency of extracting the product of Example 6, as a function of purity, using ethyl acetate (▴) or a mixture of ethyl acetate and acetone (x) using 1, 2, and 3 extractions.

### DETAILED DESCRIPTION OF THE INVENTION

Methods for preparing isatins from isonitrosoacetanilides are provided by preventing, minimizing, or eliminating the formation of undesirable by-products. Desirably, the methods prevent or minimize the formation of isatin oxime by-products.

As discussed by Kollmar et al. cited above, hydroxylamine (NH₂OH) is likely generated during the formation of isatins by the hydrolysis of isonitrosoacetanilide when reacted with a dilute acid. This generated hydroxylamine likely reacts with the strong carbonyl moiety of the isatin to form the isatin oxime. However, Kollmar did not propose a solution to prevent or eliminate this side-reaction. The inventors therefore utilized a decoy agent to prevent or minimize the formation of the isatin oxime. Formation of the oxime impurity is desirably inhibited or prevented during preparation of the isatin, during the quenching step, extraction step, or a combination thereof.

The methods include one or more of preparing an isatin in the presence of a decoy agent; quenching the reaction of preparing an isatin in the presence of a decoy agent; or extracting an isatin in the presence of a decoy agent.

A variety of isatins can be prepared and include those having the following structure: 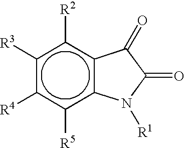
wherein, R¹ is H, OH, NH₂, C₁ to C₆ alkyl, or substituted C₁ to C₆ alkyl; R², R³, R⁴, and R⁵ are independently selected from among halogen, C₁ to C₆ alkyl, C₁ to C₆ substituted alkyl, C₃ to C₈ cycloalkyl, substituted C₃ to C₈ cycloalkyl, aryl, substituted aryl, heteroaryl, substituted heteroaryl, OR⁶, N(R⁷)₂, CON(R⁷)₂, SO₂N(R⁷)₂, and C(O)R⁸; or R² and R³; R³ and R⁴; R⁴ and R⁵; or R⁵ and R¹ are fused to form a (i) a 3 to 9 membered carbon-based saturated or unsaturated ring or (ii) a 3 to 9 membered heterocyclic ring containing in its backbone one to three heteroatoms selected from among O, S and N; R⁶ is C₁ to C₆ alkyl or C₁ to C₆ substituted alkyl; R⁷ is H, C₁ to C₆ alkyl, C₁ to C₆ substituted alkyl, or CF₃; and R⁸ is H, OH, C₁ to C₆ alkyl, substituted C₁ to C₆ alkyl, C₁ to C₆ alkoxy, substituted C₁ to C₆ alkoxy. 7-fluoroisatin can therefore be prepared.

In one embodiment, R² and R³, R³ and R⁴, or R⁴ and R⁵ are fused to form a —OCH₂CH₂O— ring. In another embodiment, R¹ is benzyl. In a further embodiment, R⁶ is benzyl.

The term “alkyl” is used herein to refer to both straight- and branched-chain saturated aliphatic hydrocarbon groups having 1 to 10 carbon atoms, desirably about 1 to 8 carbon atoms, and more desirably 1 to 6 carbon atoms. The term “cycloalkyl” is used herein to refer to an alkyl group that is cyclic in structure and has about 3 to 10 carbon atoms, desirably about 3 to 8 carbon atoms, and more desirably 5 to 8 carbon atoms.

The term “substituted alkyl” or “cycloalkyl” refers to an alkyl or cycloalkyl group having one or more substituents including, without limitation, aryl, such as phenyl, or heterocyclic, which groups can be optionally substituted. These substituents can be attached to any carbon of the alkyl or cycloalkyl group provided that the attachment constitutes a stable chemical moiety.

The term “aryl” as used herein refers to a carbocyclic aromatic system, desirably having 6 to 14 carbon atoms, which can include a single ring or multiple rings fused or linked together where at least one part of the fused or linked rings forms the conjugated aromatic system. The aryl groups can include, but are not limited to, phenyl, naphthyl, biphenyl, anthryl, tetrahydronaphthyl, phenanthryl, indene, benzonaphthyl, fluorenyl, and carbazolyl.

The term “substituted aryl” refers to an aryl group which is substituted with one or more substituents including alkyl or cycloalkyl, which groups can be optionally substituted. Desirably, a substituted aryl group is substituted with 1 to 4 substituents.

The term “heteroaryl” as used herein refers to a stable 5- to 14-membered monocyclic or multicyclic aromatic heterocyclic ring system. The heteroaryl ring has carbon atoms and one or more heteroatoms including nitrogen, oxygen, and sulfur atoms. Desirably, the heteroaryl ring has 1 to about 4 heteroatoms in the backbone of the ring. When the heteroaryl ring contains nitrogen or sulfur atoms in the backbone of the ring, the nitrogen or sulfur atoms can be oxidized.

The term “heterocyclic” refers to optionally saturated or partially saturated heterocyclic rings having 3 to 15 ring atoms, desirably 3 to 8 ring atoms, and desirably containing from 1 to 3 heteroatoms selected from among O, S and N. When the heterocyclic ring contains nitrogen or sulfur atoms in the backbone of the ring, the nitrogen or sulfur atoms can be oxidized. The term “heterocyclic” also refers to multicyclic rings in which a heterocyclic ring is fused to an aryl ring. The heterocyclic ring can be attached to the aryl ring through a heteroatom or carbon atom provided the resultant heterocyclic ring structure is chemically stable.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Oxygen-containing rings include, but are not limited to, furyl, tetrahydrofuranyl, pyranyl, pyronyl, and dioxinyl rings. Nitrogen-containing rings include, without limitation, pyrrolyl, pyrazolyl, imidazolyl, triazolyl, pyridyl, piperidinyl, 2-oxopiperidinyl, pyridazinyl, pyrimidinyl, pyrazinyl, piperazinyl, azepinyl, triazinyl, pyrrolidinyl, and azepinyl rings. Sulfur-containing rings include, without limitation, thienyl and dithiolyl rings. Mixed heteroatom containing rings include, but are not limited to, oxathiolyl, oxazolyl, thiazolyl, oxadiazolyl, oxatriazolyl, dioxazolyl, oxathiazolyl, oxathiolyl, oxazinyl, oxathiazinyl, morpholinyl, thiamorpholinyl, thiamorpholinyl sulfoxide, oxepinyl, thiepinyl, and diazepinyl rings. Fused heteroatom-containing rings include, but are not limited to, benzofuranyl, thionapthene, indolyl, benazazolyl, purindinyl, pyranopyrrolyl, isoindazolyl, indoxazinyl, benzoxazolyl, anthranilyl, benzopyranyl, quinolinyl, isoquinolinyl, benzodiazonyl, napthylridinyl, benzothienyl, pyridopyridinyl, benzoxazinyl, xanthenyl, acridinyl, and purinyl rings.

The term “substituted heterocyclic” as used herein refers to a heterocyclic group having one or more substituents including alkyl or cycloalkyl, which groups can be optionally substituted. Desirably, a substituted heterocyclic group is substituted with 1 to 4 substituents.

The term “halogen” as used herein refers to Cl, Br, F, or I groups.

The term “quenching”, or variations thereof, as used herein refers to a process of stopping a chemical reaction. With reference to the present application, the term “quenching” refers to the process of stopping the process of converting an isonitrosoacetanilide to the corresponding isatin.

The methods thereby prevent or minimize formation of isatin oximes, particularly isatin oximes of the structure: 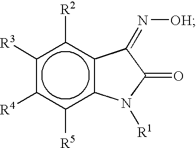
wherein, R¹-R⁵ are as defined above.

In one embodiment, an exogenous sample of the decoy agent is utilized to reduce or prevent formation of the isatin oxime. In another embodiment, the decoy agent is formed in situ from a latent decoy agent.

The term “latent decoy agent” or “latent carbonyl compound” as used herein refers to a chemical compound that forms a carbonyl functional group during a chemical reaction. Desirably, the latent decoy agent reacts with a proton (H⁺) to form a carbonyl group. More desirably, the latent decoy agent forms a carbonyl group in an acidic medium. One of skill in the art would readily be able to identify latent decoy agents useful using the teachings of the present application. Latent decoy agents include, without limitation, acetals, ketals, and bisulfite adducts of the decoy agents identified below.

The decoy agent desirably contains a carbonyl group. Alternatively, the decoy agent contains a thiocarbonyl group. In one embodiment, the decoy agent is of the structure: 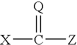
wherein, Q is O or S; X and Z are independently H, C₁ to C₆ alkyl, substituted C₁ to C₆ alkyl, aryl, substituted aryl, heteroaryl, substituted heteroaryl, C₃ to C₈ cycloalkyl, substituted C₃ to C₈ cycloalkyl, C₁ to C₁₂ alkyl(O)R², substituted C₁ to C₁₂ alkyl(O)R², C₃ to C₈ cycloalkyl C(O)R², substituted C₃ to C₈ cycloalkyl C(O)R², CY₃, COOR², or (C₁ to C₆ alkyl)OH; R² is H, C₁ to C₆ alkyl, substituted C₁ to C₆ alkyl, aryl, substituted aryl, heteroaryl, or substituted heteroaryl; and Y is halogen.

In another embodiment, the decoy agent is of the structure: 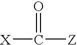
wherein, X and Z are independently H, C₁ to C₆ alkyl, substituted C₁ to C₆ alkyl, aryl, substituted aryl, heteroaryl, substituted heteroaryl, C₃ to C₈ cycloalkyl, substituted C₃ to C₈ cycloalkyl, C₁ to C₁₂ alkyl(O)R², substituted C₁ to C₁₂ alkyl(O)R², C₃ to C₈ cycloalkyl C(O)R², substituted C₃ to C₈ cycloalkyl C(O)R², CY₃, COOR², or (C₁ to C₆ alkyl)OH; R² is H, C₁ to C₆ alkyl, substituted C₁ to C₆ alkyl, aryl, substituted aryl, heteroaryl, or substituted heteroaryl; and Y is halogen. Desirably, the decoy agent has a high molecular weight.

In a further embodiment, the decoy agent or latent decoy agent is selected from among formaldehyde, paraform, formalin, acetaldehyde, propionaldehyde, acetone, 2-butanone, 2-pentanone, 3-pentanone, 4-methyl-2-pentanone, dimethoxyacetaldehyde, benzaldehyde, acetophenone, thiophenecarboxaldehyde, glyoxal, chlorals, mesoxalates, glyoxylates, pyruvates, hexafluoroacetone, diacetyl, glyoxylic acid, trioxane, diethoxymethane, dimethoxymethane, 2,2-dimethoxypropane, 1,1-dimethoxyethane, 1,1,3,3-tetramethoxypropane, diethylacetaldehyde acetal, 1,3-dioxane, 1,3-dioxolane, (CH₃)₂C(—OCH₂CH₂CH₂O—), or combinations thereof. More desirably, the decoy agent is acetone, chloral hydrate, glyoxal, or ethyl glyoxalate. In another embodiment, the decoy agent is a reducing sugar including, without limitation, glucose, lactose, and maltose.

In one embodiment, the decoy agent is added in a 1:1 ratio of isonitrosoacetanilide:decoy agent. In another embodiment, excess amounts of the decoy agent are utilized depending on the isonitrosoacetanilide utilized, amount of hydroxylamine generated, conditions of the reaction, or the degree of hydrolysis of the isonitrosoacetanilide.

The methods of preparing the isatin can be performed in the presence of a reaction solvent. However, the reaction solvent is not required. By the term “reaction solvent” is meant the solvent that is utilized to perform the reaction of converting the isonitrosoacetanilide to the isatin. A variety of reaction solvents can be utilized and are readily determined by one of skill in the art depending on the isatin being prepared, solubility of the isonitrosoacetanilide, among other factors. However, common solvents include, without limitation, toluene, isopropyl acetate, 2-butantone, 3-pentanone, or methyl isobutyl ketone.

Desirably, the isatin is prepared using a first decoy agent. The reaction can be quenched using the first decoy agent or can be extracted using a second decoy agent. Any isatin oxime prepared therein can be extracted using the first or second decoy agent or can be extracted using a third decoy agent.

Isonitrosoacetanilides that can be utilized include those having the following structure. 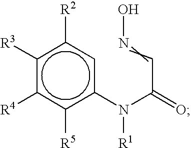
wherein, R¹-R⁵ are as defined above.

In one embodiment, the isonitrosoacetanilide is of the structure, wherein R¹-R⁵ are defined above:

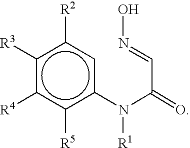

In a further embodiment, the isonitrosoacetanilide is N1-(2-fluorophenyl)-2-hydroxyiminoacetamide; 4-fluoroisonitrosoacetanilide; or 5,6,7,8-naphthyl-1-isonitrosoacetanilide; and N1-(2,4-dichlorophenyl)-2-hydroxyiminoacetamide.

The methods are also performed in the presence of an agent that cyclizes the isonitrosoacetanilide. Such cyclizing agents are known in the art and include strong acids, anhydrous hydrogen fluoride, and trifluoroborate etherate at about 90° C., among others. One of skill in the art would readily be able to select a suitable strong acid to prepare the isatins. Desirably, the strong acid is selected from among, without limitation, sulfuric acid, polyphosphoric acid, methanesulfonic acid, and combinations thereof. The cyclization is optionally performed in the presence of a chemical compound that mitigates heat generation. Desirably, the heat mitigating chemical compound does not react with the strong acid and has a boiling point that is close to the reaction temperature. More desirably, the cyclization is performed in the presence of a hydrocarbon, such as hexane, among others.

The conversion of the isonitrosoacetanilide to the isatin can be performed at room temperatures up to the reflux temperature of lowest boiling reagent in the reaction. In one embodiment, the isonitrosoacetanilide is added to a solution of the cyclizing agent, and optional reaction solvent, optionally at elevated temperatures. In another embodiment, the cyclizing agent and optional reaction solvent are mixed with the isonitrosoacetanilide, and optionally heated to elevated temperatures. Desirably, the latter method is performed on large scale reactions. In a further embodiment, the isonitrosoacetanilide is added to a solution of the cyclizing agent, decoy agent, and optional reaction solvent, optionally at elevated temperatures. In another embodiment, the cyclizing agent, decoy agent, and optional reaction solvent are mixed with the isonitrosoacetanilide, and optionally heated to elevated temperatures.

After conversion of the isonitrosoacetanilide to the isatin, the reaction mixture is quenched to stop the reaction from further proceeding and thereby prevent the formation of undesirable side-products. The term “quenching solvent” as used herein is meant to describe a solvent utilized to stop a reaction from further proceeding, i.e., to stop the reaction whereby the isatin is formed. The quenching solvent includes one or more decoy agents alone or in combination with other solvents. Other quenching solvents that can be utilized in combination with the decoy agent includes, without limitation, water, toluene, isopropyl acetate, among others. Typically, the decoy agent is the quenching solvent and is used in combination with water.

After quenching, the reaction mixture can be subjected to several steps to isolate the isatin, i.e., “work-up” steps, including extractions using an extraction solvent. The term “extraction solvent” is meant to describe a solvent utilized after the isatin is formed and during work-up of the reaction. The extraction solvent can readily be selected by one of skill in the art and includes, without limitation, the decoy agent, toluene, isopropyl acetate, and combinations thereof. Desirably, the extraction solvent is the decoy agent. More desirably, the extraction solvent is a water-immiscible decoy agent. Typically, one, two, three or more extractions are utilized to isolate the isatin from the reaction mixture.

The inventors have found that when the isatin is extracted in the absence of a decoy agent, solid material and emulsions were formed. However, when a decoy agent is utilized during the extraction, the inventors found that higher yields of the isatin and lower yields of the isatin oxime were obtained. The use of a decoy agent during extraction also permits fewer extraction steps than when the extractions are performed in the absence of the decoy agent.

In one embodiment, methods of preparing an isatin are provided and include adding an isonitrosoacetanilide to a solution of a strong acid, a decoy agent, and an optional reaction solvent. The solution is typically heated to elevated temperatures prior to addition of the isonitrosoacetanilide.

In another embodiment, a method is provided for preventing or minimizing the formation of isatin oximes, including preparing an isatin from an isonitrosoacetanilide in a first decoy agent comprising a carbonyl group; and extracting the isatin in a second decoy agent comprising a carbonyl group. In one example, the first and second decoy agents are the same. In another example, the first and second decoy agents are different.

In a further embodiment, methods of isolating an isatin from a reaction mixture are provided and include quenching the reaction using a decoy agent.

In still a further embodiment, methods of isolating an isatin from a reaction mixture are provided and include extracting the isatin from the reaction mixture using a decoy agent.

In another embodiment, methods of preparing an isatin are provided and include mixing an isonitrosoacetanilide, a strong acid, and an optional reaction solvent; optionally heating the solution to elevated temperatures; and quenching the solution with a decoy agent.

In yet another embodiment, methods of preparing an isatin are provided and include mixing an isonitrosoacetanilide, a strong acid, and an optional reaction solvent; optionally heating the solution to elevated temperatures; and extracting the isatin using a decoy agent.

In a further embodiment, methods of preparing an isatin are provided and include mixing an isonitrosoacetanilide, a strong acid, and an optional reaction solvent; optionally heating the solution to elevated temperatures; quenching the solution with a decoy agent; and extracting the isatin using a decoy agent.

In still another embodiment, methods of preparing an isatin are provided and include mixing an isonitrosoacetanilide, sulfuric acid, and hexane; heating the solution to elevated temperatures, desirably to the reflux temperature of hexane; maintaining elevated temperatures until the reaction was complete; and quenching the reaction with a decoy agent.

In a further embodiment, methods of preparing an isatin are provided and include mixing an isonitrosoacetanilide, sulfuric acid, and hexane; heating the solution to elevated temperatures, desirably to the reflux temperature of hexane; maintaining elevated temperatures until the reaction was complete; quenching the reaction with a decoy agent; and extracting the isatin using a decoy agent.

In yet a further embodiment, methods of preparing 7-fluoroisatin are provided and include reacting isonitrosoacetanilide and a strong acid and quenching said reaction using a decoy agent.

The isatins prepared as described herein are useful as intermediates in the preparation of a variety of compounds, including pharmaceutical compounds. For example, 2-(4-chlorobenzyl)-3-hydroxy-7,8,9,10-tetrahydrobenzo[h]quinoline-4-carboxylic acid and (1R)-5-cyano-1,3,4,9-tetrahydro-8-methyl-1-propyl-pyrano[3,4-b]indole-1-acetic acid can be prepared according to the methods herein via the corresponding 6,7,8,9-tetrahydro-1H-benz[g]isatin and 4-bromo-7-methylisatin, respectively.

Also included is the preparation of 5-(7-fluoro-3,3-dimethyl-2-oxo-2,3-dihydro-1H-indol-5-yl)-1-methyl-1H-pyrrole-2-carbonitrile using one or more of the decoy agents identified above. See, Scheme 1.

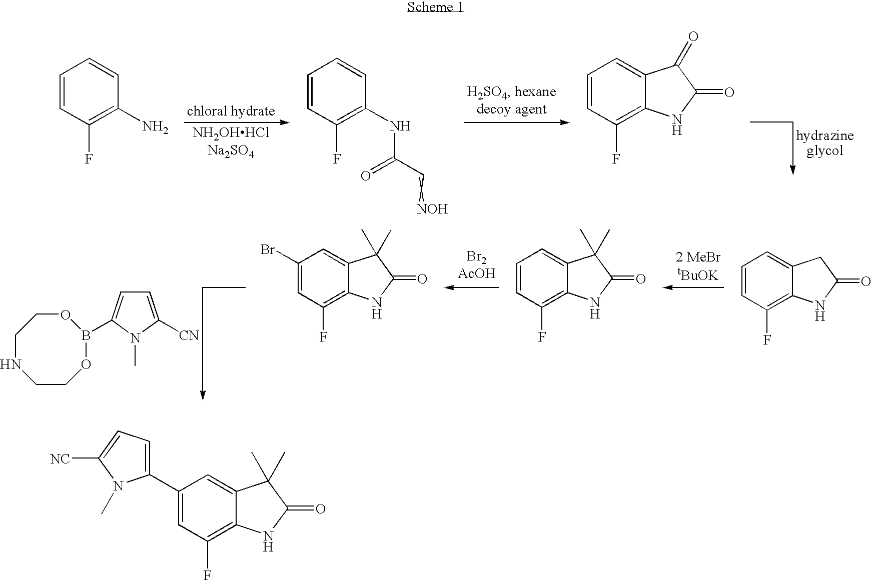

The preparation of 5-(7-fluoro-3,3-dimethyl-2-oxo-2,3-dihydro-1H-indol-5-yl)-1-methyl-1H-pyrrole-2-carbonitrile thereby includes reacting 2-fluoroaniline, chloral hydrate, and hydroxylamine hydrochloride; (b) reacting the product of step (a) with sulfuric acid in the presence of hexane and a decoy agent; (c) reacting the product of step (b) with hydrazine and glycol; (d) reacting the product of step (c) with 2 equivalents of methylbromide; (e) reacting the product of step (d) with bromine; and (f) reacting the product of step (e) with 5-[1,3,6,2]dioxazaborocyan-2-yl-1-methyl-1H-pyrrole-2-carbonitrile. The process can also include quenching the reaction of step (b) with a decoy agent, extracting the product of step (b) using a decoy agent, or a combination thereof.

Also included are methods of preparing 5-(7-fluoro-3,3-dimethyl-2-oxo-2,3-dihydro-1H-indol-5-yl)-1-methyl-1H-pyrrole-2-carbonitrile, including (a) reacting 2-fluoroaniline, chloral hydrate, and hydroxylamine hydrochloride; (b) reacting the product of step (a) with sulfuric acid in the presence of hexane; (c) quenching the reaction of step (b) using a decoy agent; (d) reacting the product of step (c) with hydrazine and glycol; (e) reacting the product of step (d) with 2 equivalents of methylbromide; (f) reacting the product of step (e) with bromine; and (g) reacting the product of step (f) with 5-[1,3,6,2]dioxazaborocyan-2-yl-1-methyl-1H-pyrrole-2-carbonitrile.

Further included are methods of preparing 5-(7-fluoro-3,3-dimethyl-2-oxo-2,3-dihydro-1H-indol-5-yl)-1-methyl-1H-pyrrole-2-carbonitrile, including (a) reacting 2-fluoroaniline, chloral hydrate, and hydroxylamine hydrochloride; (b) reacting the product of step (a) with sulfuric acid in the presence of hexane; (c) extracting the product of step (b) using a decoy agent; (d) reacting the product of step (c) with hydrazine and glycol; (e) reacting the product of step (d) with 2 equivalents of methylbromide; (f) reacting the product of step (e) with bromine; and (g) reacting the product of step (f) with 5-[1,3,6,2]dioxazaborocyan-2-yl-1-methyl-1H-pyrrole-2-carbonitrile.

Isatin oximes can also be prepared and include reacting an isonitrosoacetanilide with hydroxylamine or salt thereof. Hydroxylamine salts that can be utilized include, without limitation, hydroxylamine hydrochloride, hydroxylamine sulfate, hydroxylamine phosphate, and hydroxylamine nitrate. See, Scheme 2.

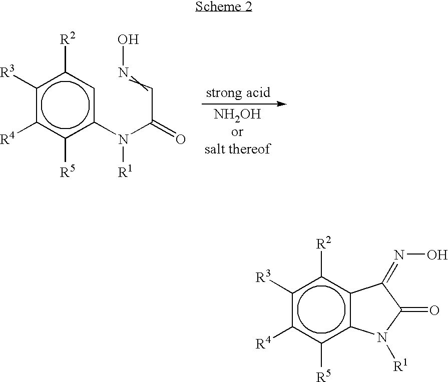

Also provided are kits or packages to prepare the isatins or isatin oximes. When the isatin is the desired product, the kits can include the isonitrosoacetanilide and decoy agent. When the isatin oxime is the desired product, the kits can include the isonitrosoacetanilide and hydroxylamine, or salt thereof. The kits can also contain other reagents useful in preparing the isatins and include solvents and strong acids. The kits can optionally include other reagents such as strong acids.

The kit can further contain instructions for performing the reactions. Also provided in a kit can be other suitable chemicals, disposable gloves, decontamination instructions, applicator sticks or containers, and sample preparator cups.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

### EXAMPLES

### Example 1

### Quench and Extraction of 7-fluoroisatin with Toluene Containing Acetone

A 500-mL flask fitted with mechanical stirrer, thermocouple, and addition funnel was charged with concentrated sulfuric acid (158 mL) and heated to 73-76° C. Milled N1-(2-fluorophenyl)-2-hydroxyiminoacetamide (49 g) was added in portions within an hour. The heating was continued for additional 0.5 hour. The dark-reddish mixture was slowly added to a 3-L flask containing cooled water (0.79 L), sodium sulfate (49 g), toluene (516 mL) and acetone (327 mL). The mixture was stirred for 15 hours at ambient temperature. The phases were separated and the aqueous phase was extracted with toluene (2×500 mL). The combined organic extracts were concentrated on a rotovap, water (250 mL) was added and the resulted slurry was filtered on a Buchner funnel and the solids were washed with water (200 mL). The wet cake was dried in a vacuum oven at 60° C. for 18 hours to give 7-fluoroisatin (18 g, 40.4% yield, 85% purity, 14% oxime by-product).

### Example 2

### Quench and Extraction of 7-fluoroisatin with Isopropyl Acetate Containing Acetone

A 500-mL flask fitted with mechanical stirrer, thermocouple, and addition funnel was charged with conc. sulfuric acid (158 mL) and heated to 73-76° C. Milled N1-(2-fluorophenyl)-2-hydroxyiminoacetamide (49 g) was added in portions within an hour. The heating was continued for additional 0.5 hour. The dark-reddish mixture was slowly added to a 3-L flask containing cooled water (0.79 L), sodium sulfate (49 g), isopropyl acetate (514 mL) and acetone (327 mL). The mixture was stirred for 15 hours at ambient temperature. The phases were separated and the aqueous phase was extracted with isopropyl acetate (2×500 mL). The combined organic extracts were concentrated on a rotovap, water (250 mL) was added and the resulted slurry was filtered on a Buchner funnel and the solids were washed with water (200 mL). The wet cake was dried in a vacuum oven at 60° C. for 18 hours to give 7-fluoroisatin (15.6 g, 35% yield, 96% purity, 3% oxime by-product).

### Example 3

### Comparative Reaction Using Extraction with Isopropyl Acetate

A 500-mL flask fitted with mechanical stirrer, thermocouple, and addition funnel was charged with concentrated sulfuric acid (200 mL) and heated to 73-76° C. Milled N1-(2-fluorophenyl)-2-hydroxyiminoacetamide (49 g) was added in portions within an hour. The heating was continued for additional 0.5 hour. The dark-reddish mixture was slowly added to a 3-L flask containing cooled water (1.5 L) and isopropyl acetate (0.40 L). Separation of the phases and additional extraction with isopropyl acetate (4×200 mL) gave dark, organic phase that was evaporated and triturated with hexane to give an orange solid (23.5 g, 43% yield; 17.3% oxime side-product).

### Example 4

### Formation of 7-fluoroisatin Oxime in the Quenched Reaction Mixture with No Additives

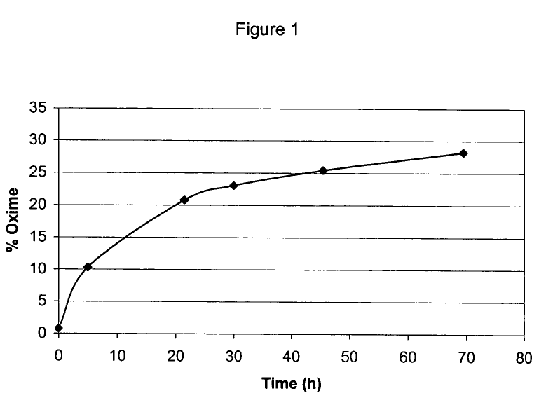

Milled N1-(2-fluorophenyl)-2-hydroxyiminoacetamide (6.0 g, 33 mmol) was added in portions into a stirred and heated concentrated sulfuric acid (20 mL). A sample of the reaction mixture was diluted with water and acetonitrile, and monitored by high performance liquid chromatography (HPLC) over time (FIG. 1).

These data illustrate that the isatin oxime by-product is formed without use of a decoy agent present during the reaction or work-up in water/acetonitrile and that the concentration of the oxime increases over time.

### Example 5

### Suppression of the Formation of 7-fluoroisatin Oxime in the Presence of Various Carbonyl-containing Additives

About 1 mL of the reaction mixture from Example 4 was transferred into Bohdan's MiniBlock® tubes containing solution (5 mL each) prepared from sodium sulfate (6.0 g) and water (75 mL) and isopropyl acetate (3 mL). Each tube contained an additive (about 5 mmol) as listed in Table 1. Appropriate samples were withdrawn after 1 and 37 hours of stirring and analyzed by HPLC.

TABLE 1

% oxime

| Decoy Agent | after 1 hour | after 37 hours |
|---|---|---|
| None | 5.1 | 39.6 |
| Acetone (80 mol %) | 0.8 | 10.8 |
| Chloral hydrate | 3.7 | 35.5 |
| Glyoxal | 1.4 | 5.2 |
| Ethyl glyoxalate | 1.1 | 10.1 |

These data illustrate that less isatin oxime by-product was produced when a decoy agent was present during the work-up procedure. However, glyoxal was the most successful over long periods of time in preventing oxime formation.

### Example 6

### Quench and Extraction of 7-fluoroisatin with Isopropyl Acetate Containing Acetone

A 1-L flask was charged with N1-(2-fluorophenyl)-2-hydroxyiminoacetamide (55 g, 90% strength), hexane (303 mL), and concentrated sulfuric acid (291 g, 158 mL) to give a suspension. The suspension was heated to 69° C. over 10 minutes and hexane was distilled off. A dark thick reaction mixture resulted as the temperature reached 75-88° C. The mixture was stirred for 1 hour at 75-88° C.

After cooling to 20-25° C., the solution was transferred, over 1 hour, to a 3-L flask containing pre-cooled 5° C. water (700 mL) containing sodium sulfate (68 g), isopropyl acetate (500 mL), and acetone (176 mL). The temperature was controlled at about 35° C. by adjusting the addition rate. The 1-L flask was rinsed with water (60 mL) and isopropyl acetate (92 mL). The top organic layer was separated and the aqueous layer was extracted with isopropyl acetate (3×500 mL). The organic extracts were concentrated on rotary evaporator to a volume of 150-200 mL. Water (300 mL) was added and the mixture was again concentrated on a rotary vapor at 50° C. under vacuum to remove isopropyl acetate. The precipitated solid was filtered on a Buchner funnel, rinsed with water (200 mL) and dried in a vacuum oven for 6 hours at 50° C.

Yield 23.8 g (47.7%; 96.1% purity, 2.2% oxime by HPLC area).

### Example 7

### Quench and Extraction of 7-fluoroisatin with N-methyl Isobutyl Ketone, Water, and Acetone

N1-(2-Fluorophenyl)-2-hydroxyiminoacetamide (3.12 g) was added in portions into stirred and heated concentrated sulfuric acid (10 mL) over 90 minutes. About 1 mL of the reaction mixture was transferred into MiniBlock XT® tubes, each containing water (5 mL), methyl isobutyl ketone (3 mL) and a decoy agent identified in Table 2. Samples were withdrawn after 24 hours of stirring and analyzed by LC/MS.

TABLE 2

| Decoy agent | Amount added | % Oxime |
|---|---|---|
| None | — | 22.7 |
| Acetone | 2 mL | 3.0 |
| D-glucose | 1 g | 13.4 |
| 37% Formaldehyde | 2 mL | 1.9 |
| Diethoxymethane | 2 mL | 0 |
| 2-(Trifluoromethyl)propionaldehyde | 1 g | 0.6 |

These data illustrate that the decoy agent reduces the formation of isatin oximes during formation of isatins from isonitrosoacetanilides.

### Example 8

### Quench and Extraction of 5,7-dichloroisatin with Isopropyl Acetate and Water Containing Glyoxal Disodium Bisulfite Adduct, Glyoxal, and Glyoxylic Acid

N1-(2,4-Dichlorophenyl)-2-hydroxyiminoacetamide (1.0 g) was added in portions into stirred and heated concentrated sulfuric acid (3 mL) over 40 minutes. The mixture was kept at 68° C. for additional 40 minutes. The reaction mixture was distributed into vials placed in Chemglass' Pie-Block™ holder, each vial separately containing water (1 mL), isopropyl acetate (0.5 mL), and the decoy agents (i) glyoxal disodium bisulfite adduct (0.108 g), (ii) glyoxal (40% in water, 0.5 mL), and (iii) glyoxylic acid (50% in water, 0.5 mL). The samples were stirred overnight, diluted with isopropyl acetate (0.5 mL each) and vortexed. Appropriate aliquots were withdrawn and analyzed by GC/MS.

In addition to the starting material and some unidentified products, all samples contained isatin (6.4, 8.4, and 5.7%, respectively) and no oxime.

### Example 9

### Extraction of 7-fluoroisatin with a Decoy Agent

N1-(2-fluorophenyl)-2-hydroxyiminoacetamide was heated in sulfuric acid to form 7-fluoroisatin as described in Example 6. A portion (45 g) of the reaction mixture of Example 6 was quenched into a solution of isopropyl acetate (52 g), water (83 g), sodium sulfate (8.1 g) and acetone (16.7 g). The phases were separated, and the lower aqueous phase was removed. The upper organic phase and the rag layer were separated, the upper organic phase was split in half, and the rag layer was split in half. Each half of the rag layer was combined with a half of the organic layer to give two equal portions.

Further extractions of these two equal portions by ethyl acetate were the compared with further extractions of the other portion by ethyl acetate containing a decoy agent. Specifically, the 7-fluoroisatin in the first portion was extracted with a series of three washes of ethyl acetate (3×60 mL ethyl acetate). Each wash was attended by interfacial rag layers and then analyzed by HPLC.

The 7-fluoroisatin in the second portion was extracted with a series of washes (3×60 mL), each wash using a mixture of ethyl acetate (54 mL) and acetone (15 mL). Each wash was cleaned without an interfacial rag layer. Each wash was analyzed by HPLC.

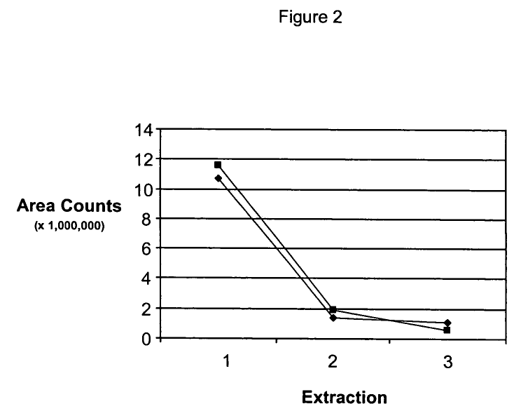

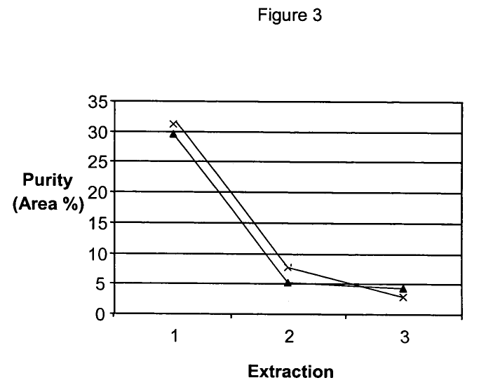

FIGS. 2 and 3 illustrate that the presence of acetone during extraction of the 7-fluoroisatin makes the extraction more efficient. Specifically, when a mixture of ethyl acetate and acetone is used, the area count, which is proportional to the amount of 7-fluoroisatin, and purity are higher in the first and second extractions. However, very little 7-fluoroistain was present in the third extraction, which resulted in a decrease in area count and purity as illustrated by FIGS. 2 and 3. In summary, the first and second extractions using a combination of ethyl acetate and acetone were sufficient to recover the 7-fluoroisatin in good area counts and purities.

When ethyl acetate alone was used for the extractions, the third extraction showed about the same area count and purity as the second extraction.

Thus, the inventors found that when the 7-fluoroisatin was extracted using a mixture of ethyl acetate and acetone, two extractions were sufficient to recover the product. However, when 7-fluoroisatin was extracted in the absence of acetone, three extractions were required to efficiently extract the product. Therefore, the presence of a decoy agent, i.e., acetone, during the extraction makes extraction of the isatin more efficient and with good isatin purities.

All publications cited in this specification are incorporated herein by reference herein. While the invention has been described with reference to a particularly preferred embodiment, it will be appreciated that modifications can be made without departing from the spirit of the invention. Such modifications are intended to fall within the scope of the appended claims.

What is claimed Is:

1. A method for preventing or minimizing the formation of isatin oximes, comprising preparing an isatin from an isonitrosoacetanilide in the presence of a decoy agent comprising a carbonyl group.

2. The method according to claim 1, further comprising extracting said isatin in said decoy agent.

3. The method according to claim 2, wherein said extraction further comprises a solvent.

4. The method according to claim 3, wherein said solvent is isopropyl acetate, 2-butantone, 3-pentanone, or methyl isobutyl ketone.

5. The method according to claim 1, wherein said isatin is of the structure: 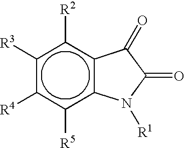
wherein:
R¹ is H, OH, NH₂, C₁ to C₆ alkyl, or substituted C₁ to C₆ alkyl;
R², R³, R⁴, and R⁵ are independently selected from the group consisting of halogen, C₁ to C₆ alkyl, C₁ to C₆ substituted alkyl, C₃ to C₈ cycloalkyl, substituted C₃ to C₈ cycloalkyl, aryl, substituted aryl, heteroaryl, substituted heteroaryl, OR⁶, N(R⁷)₂, CON(R⁷)₂, SO₂N(R⁷)₂, NO₂, CN, and C(O)R⁸; or
R² and R³; R³ and R⁴; R⁴ and R⁵; or R⁵ and R¹ are fused to form a (i) a 3 to 14 membered carbon-based saturated or unsaturated ring or (ii) a 3 to 14 membered heterocyclic ring containing in its backbone one to three heteroatoms selected from the group consisting of O, S and N;
R⁶ is CF₃, C₁ to C₆ alkyl or C₁ to C₆ substituted alkyl;
R⁷ is H, C₁ to C₆ alkyl, C₁ to C₆ substituted alkyl, CF₃, C(O)R⁹, NC(O)R⁹, or (C₁ to C₆ alkyl)NC(O)R⁹; or
two of R⁷ are fused to form a -A-(CH₂)ₙ-A- ring;
n is 1 to 6;
A are independently selected from the group consisting of O, S, and N;
R⁸ is H, OH, C₁ to C₆ alkyl, substituted C₁ to C₆ alkyl, C₁ to C₆ alkoxy, substituted C₁ to C₆ alkoxy; and
R⁹ is CF₃, C₁ to C₆ alkyl, substituted C₁ to C₆ alkyl, NHC(O)CF₃, and CH₂CH₂NHC(O)CF₃.

6. The method according to claim 5, wherein:
R², R³, R⁴, and R⁵ are independently selected from the group consisting of halogen, C₁ to C₆ alkyl, C₁ to C₆ substituted alkyl, C₃ to C₈ cycloalkyl, substituted C₃ to C₈ cycloalkyl, aryl, substituted aryl, heteroaryl, substituted heteroaryl, OR⁶, N(R⁷)₂, CON(R⁷)₂, SO₂N(R⁷)₂, and C(O)R⁸; or
R² and R³; R³ and R⁴; R⁴ and R⁵; or R⁵ and R¹ are fused to form a (i) a 3 to 9 membered carbon-based saturated or unsaturated ring or (ii) a 3 to 9 membered heterocyclic ring containing in its backbone one to three heteroatoms selected from the group consisting of O, S and N;
R⁶ is C₁ to C₆ alkyl or C₁ to C₆ substituted alkyl; and
R⁷ is H, C₁ to C₆ alkyl, C₁ to C₆ substituted alkyl, or CF₃.

7. The method according to claim 5, wherein R² and R³; R³ and R⁴; or R⁴ and R⁵ are fused to form —OCH₂CH₂O—.

8. The method according to claim 1, wherein said isatin is 7-fluoroisatin.

9. The method according to claim 1, wherein said isatin oxime is of the structure: 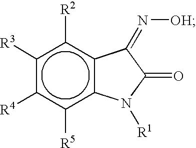
wherein:
R¹-R⁵is H, OH, NH₂, C₁ to C₆ alkyl, or substituted C₁ to C₆ alkyl;
R², R³, R⁴, and R⁵ are independently selected from the group consisting of halogen, C₁ to C₆ alkyl, C₁ to C₆ substituted alkyl, C₃ to C₈ cycloalkyl, substituted C₃ to C₈ cycloalkyl, aryl, substituted aryl, heteroaryl, substituted heteroaryl, OR⁶, N(R⁷)₂, CON(R⁷)₂, SO₂N(R⁷)₂, and C(O)R⁸; or
R² and R³; R³ and R⁴; R⁴ and R⁵; or R⁵and R¹ are fused to form a (i) a 3 to 14 membered carbon-based saturated or unsaturated ring or (ii) a 3 to 14 membered heterocyclic ring containing in its backbone one to three heteroatoms selected from the group consisting of O, S and N;
R⁶ is C₁ to C₆ alkyl or C₁ to C₆ substituted alkyl;
R⁷is H, C₁ to C₆ alkyl, C₁ to C₆ substituted alkyl, or CF₃; and
R⁸is H, OH, C₁ to C₆ alkyl, substituted C₁ to C₆ alkyl, C₁ to C₆ alkoxy, substituted C₁ to C₆ alkoxy.

10. The method according to claim 1, wherein said decoy agent is of the structure: 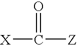
wherein:
X and Z are, independently, H, C₁ to C₆ alkyl, substituted C₁ to C₆ alkyl, aryl, substituted aryl, heteroaryl, substituted heteroaryl, C₃ to C₈ cycloalkyl, substituted C₃ to C₈ cycloalkyl, C₁ to C₁₂ alkyl(O)R², substituted C₁ to C₁₂ alkyl(O)R², C₃ to C₈ cycloalkyl C(O)R², substituted C₃ to C₈ cycloalkyl C(O)R², CY₃, COOR², or (C₁ to C₆)OH;
R² is H, C₁ to C₆ alkyl, substituted C₁ to C₆ alkyl, aryl, substituted aryl, heteroaryl, or substituted heteroaryl; and
Y is halogen.

11. The method according to claim 1, wherein said decoy agent is selected from the group consisting of formaldehyde, paraform, formalin, acetaldehyde, propionaldehyde, acetone, 2-butanone, 2-pentanone, 3-pentanone, 4-methyl-2-pentanone, dimethoxyacetaldehyde, benzaldehyde, acetophenone, thiophenecarboxaldehyde, glyoxal, chlorals, mesoxalates, glyoxylates, pyruvates, hexafluoroacetone, diacetyl, glyoxylic acid, a corresponding hydrate, and combinations thereof.

12. The method according to claim 1, wherein said decoy agent is acetone, chloral hydrate, glyoxal, or ethyl glyoxalate.

13. The method according to claim 1, wherein said decoy agent is formed during said method.

14. The method according to claim 13, wherein said decoy agent is formed from a latent decoy agent.

15. The method according to claim 13, wherein said decoy agent is a reducing sugar.

16. The method according to claim 15, wherein said reducing sugar is selected from the group consisting of glucose, lactose, and maltose.

17. The method according to claim 14, wherein said latent decoy agent is an acetal, ketal, ketal thio-derivative, or bisulfite addition compound.

18. The method according to claim 17, wherein said ketal is trioxane, diethoxymethane, dimethoxymethane, 2,2-dimethoxypropane, 1,1-dimethoxyethane, 1,1,3,3-tetramethoxypropane, diethylacetaldehyde acetal, 1,3-dioxane, 1,3-dioxolane, or (CH₃)₂C(—OCH₂CH₂CH₂O—).

19. The method according to claim 1, wherein said isonitrosoacetanilide is of the structure: 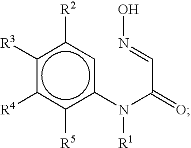
wherein:
R¹ is H, OH, NH₂, C₁ to C₆ alkyl, or substituted C₁ to C₆ alkyl;
R², R³, R⁴, and R⁵ are independently selected from the group consisting of halogen, C₁ to C₆ alkyl, C₁ to C₆ substituted alkyl, C₃ to C₈ cycloalkyl, substituted C₃ to C₈ cycloalkyl, aryl, substituted aryl, heteroaryl, substituted heteroaryl, OR⁶, N(R⁷)₂, CON(R⁷)₂, SO₂N(R⁷)₂, and C(O)R⁸; or
R² and R³; R³ and R⁴; R⁴ and R⁵; or R⁵ and R¹ are fused to form a (i) a 3 to 14 membered carbon-based saturated or unsaturated ring or (ii) a 3 to 14 membered heterocyclic ring containing in its backbone one to three heteroatoms selected from the group consisting of O, S and N;
R⁶ is C₁ to C₆ alkyl or C₁ to C₆ substituted alkyl;
R⁷ is H, C₁ to C₆ alkyl, C₁ to C₆ substituted alkyl, or CF₃; and
R⁸ is H, OH, C₁ to C₆ alkyl, substituted C₁ to C₆ alkyl, C₁ to C₆ alkoxy, substituted C₁ to C₆ alkoxy.

20. The method according to claim 1, wherein said isonitrosoacetanilide is selected from the group consisting of N1-(2-fluorophenyl)-2-hydroxyiminoacetamide; 4-fluoroisonitrosoacetanilide; and 5,6,7,8-napthyl-1-isonitrosoacetanilide; and N1-(2,4-dichlorophenyl)-2-hydroxyiminoacetamide.

21. The method according to claim 1, wherein said isatin is formed in the presence of a strong acid.

22. The method according to claim 21, wherein said strong acid is selected from the group consisting of sulfuric acid, polyphosphoric acid, methanesulfonic acid, and combinations thereof.

23. A product prepared by the method of claim 1.

24. A method for preventing or minimizing the formation of isatin oximes, comprising quenching the reaction for forming an isatin from an isonitrosoacetanilide in the presence of a decoy agent comprising a carbonyl group.

25. A method of preparing 7-fluoroisatin by reacting 2-fluoroisonitrosoacetanilide with a strong acid and quenching said reaction with a decoy agent comprising a carbonyl group.

26. A method for preventing or minimizing the formation of isatin oximes, comprising
(i) preparing an isatin from an isonitrosoacetanilide in a first decoy agent comprising a carbonyl group; and
(ii) extracting said isatin in a second decoy agent comprising a carbonyl group.

27. The method according to claim 26, wherein said first and second decoy agents are the same.

28. The method according to claim 26, wherein said first and second decoy agents are different.

29. A method for preparing an isatin oxime comprising reacting an isonitrosoacetanilide with a hydroxylamine or salt thereof.

30. The method according to claim 29, wherein said hydroxylamine salt is selected from the group consisting of hydroxylamine hydrochloride, hydroxylamine sulfate, hydroxylamine phosphate, and hydroxylamine nitrate.

31. A method of preparing 5-(7-fluoro-3,3-dimethyl-2-oxo-2,3-dihydro-1H-indol-5-yl)-1-methyl-1H-pyrrole-2-carbonitrile, comprising:
(a) reacting 2-fluoroaniline, chloral hydrate, and hydroxylamine hydrochloride;
(b) reacting the product of step (a) with sulfuric acid in the presence of hexane and a decoy agent;
(c) reacting the product of step (b) with hydrazine and glycol;
(d) reacting the product of step (c) with 2 equivalents of methylbromide;
(e) reacting the product of step (d) with bromine; and
(f) reacting the product of step (e) with 5-[1,3,6,2]dioxazaborocyan-2-yl-1-methyl-1H-pyrrole-2-carbonitrile.

32. A method of preparing 5-(7-fluoro-3,3-dimethyl-2-oxo-2,3-dihydro-1H-indol-5-yl)-1-methyl-1H-pyrrole-2-carbonitrile, comprising:
(a) reacting 2-fluoroaniline, chloral hydrate, and hydroxylamine hydrochloride;
(b) reacting the product of step (a) with sulfuric acid in the presence of hexane;
(c) quenching the reaction of step (b) using a decoy agent;
(d) reacting the product of step (c) with hydrazine and glycol;
(e) reacting the product of step (d) with 2 equivalents of methylbromide;
(f) reacting the product of step (e) with bromine; and
(g) reacting the product of step (f) with 5-[1,3,6,2]dioxazaborocyan-2-yl-1-methyl-1H-pyrrole-2-carbonitrile.

33. A method of preparing 5-(7-fluoro-3,3-dimethyl-2-oxo-2,3-dihydro-1H-indol-5-yl)-1-methyl-1H-pyrrole-2-carbonitrile, comprising:
(a) reacting 2-fluoroaniline, chloral hydrate, and hydroxylamine hydrochloride;
(b) reacting the product of step (a) with sulfuric acid in the presence of hexane;
(c) extracting the product of step (b) using a decoy agent;
(d) reacting the product of step (c) with hydrazine and glycol;
(e) reacting the product of step (d) with 2 equivalents of methylbromide;
(f) reacting the product of step (e) with bromine; and
(g) reacting the product of step (f) with 5-[1,3,6,2]dioxazaborocyan-2-yl-1-methyl-1H-pyrrole-2-carbonitrile.
